(19) United States
(12) Patent Application Publication
(10) Pub. No.: US 2014/0287998 A1
(43) Pub. Date: Sep. 25, 2014

(54) GLUCAGON FORMULATIONS

(71) Applicant: Enject, Inc., Battle Ground, WA (US)

(72) Inventors: Richard W. Rylander, JR., Battle Ground, WA (US); Solomon Tse, San Diego, CA (US)

(21) Appl. No.: 14/357,174

(22) PCT Filed: Dec. 7, 2012
(86) PCT No.: PCT/US2012/068402

§ 371 Date: May 8, 2014

Related U.S. Application Data
(60) Provisional application No. 61/568,711, filed on Dec. 9, 2011.

(51) Int. Cl. A61K 9/08; A61K 38/26

(52) U.S. Cl. CPC A61K 9/08; A61K 38/26
USPC 514/11.7

(57) ABSTRACT

Glucagon is formulated in hydrochloric acid buffered, mannitol containing formulations that can be readily lyophilized and rapidly reconstituted for use in dual chamber cartridges and auto-injector device technology.

### BACKGROUND OF THE INVENTION

The present invention provides glucagon formulations and devices containing those formulations that facilitate administration. The present invention therefore relates to the fields of pharmacology, medicine, and medical device technology.

Diabetes is a chronic, lifelong disease associated with high levels of glucose in the blood. Diabetes is widespread; there are estimated (http://www.diabetes.org/diabetes-basics/diabetes-statistics/) to be more than 23 million patients with type 1 and type 2 diabetes in the United States with global prevalence estimates exceeding 240 million in 2006 (http://www.dagc.org/diastatsglobal.asp). The number of patients continues to increase at a rate that far exceeds the population growth. Many patients with diabetes self administer insulin to control their blood glucose levels. However, insulin administration can cause a rapid drop in blood sugar leading to a very low blood sugar level (known as severe hypoglycemia), which is a serious, life-threatening condition. Severe Hypoglycemia (also known as Emergency Hypoglycemia) can be treated by administration of an injection of glucagon. By definition, hypoglycemia is defined as severe when the assistance of a third party is necessary. Many diabetics on insulin accordingly keep a supply of glucagon available at all times.

Two existing products, the Glucagon Emergency Kit (marketed by Eli Lilly and Company) and the GlucaGen Hypo-kit (marketed by Novo-Nordisk), are available to meet this need of diabetic patients for injectable glucagon. These products are essentially identical. A vial containing 1 mg of lyophilized human recombinant glucagon and a syringe pre-filled with 1 ml of diluent are packaged in a hard plastic outer case. These products suffer from numerous problems.

Most of these problems are related to the complex operations and the relatively long time needed to prepare and administer the drug. In a severe hypoglycemic situation, the diabetic patient may lose motor control as well as coordination and is unable to self administer any treatment. A second person must locate the glucagon kit, open it, remove the vial and syringe, remove the cover of the vial, remove the cover of the needle on the syringe, insert the syringe needle into the vial, and aspirate the contents of the syringe into the vial. The syringe needle can be removed or left in place while the solvent from the syringe and the lyophilized glucagon powder in the vial are being mixed. Both manufacturers recommend that the vial be swirled or rotated to assure the powder dissolves. This process may take as much as 30 seconds or longer. At that point, the vial should be inverted (the needle needs to be reinserted if it was removed during the dissolution process) and the contents of the vial drawn back into the syringe, the syringe needle removed from the vial, and the injection given in a muscular region. Following the injection the needle is withdrawn, the needle cover replaced, and the syringe, vial, and plastic case should be properly discarded. Given the emergency nature of the condition, since time is of the essence, the person preparing and administering the injection can make errors and in any event has to make decisions under great stress. In this regard, as per the FDA approved label for both existing products, the 1 mg dose is only for administration to an adult; a child should be administered only 0.5 mg but there is no simple way to limit the amount of the injection. Bent needles, dropped vials and syringes, secondary needle sticks, improper mixing, failure to administer the recommended dose, and other errors have been common over the 20+ years of use of this type of system.

During this 20+ year period, medical device technology has advanced greatly, but these advances have not helped improve glucagon delivery technology. One remarkable advance is the dual chamber cartridge, commonly known as the Vetter V-LK system marketed by Vetter Pharma. This system was specially designed to allow the lyophilization of a drug in one-half of the unit (first chamber or front chamber) with a solvent to be placed in a separate portion (the other half, second chamber or back chamber) of the cartridge separated by a movable elastomeric barrier and sealed on the two outer ends. Several human growth hormone products are marketed using the cartridge system as well as Pegylated Interferon (Schering Plough) and Edex (Schwartz Pharma). This technology is also being used for a variety of drugs in development, including Bydureon (marketed by Amylin). The equipment, system and process requirements are such that Vetter Pharma is the predominant manufacturer globally and is currently the sole supplier (directly or through licensing agreements) for approved and marketed dual chamber cartridges in the United States. The Vetter V-LK system allows for up to 1 ml of drug/solvent to be delivered. Boehringer-Ingelheim has also begun marketing similar or identical technologies to those used by Vetter Pharma.

Dual chamber cartridges typically utilize lyophilized (freeze dried) drugs and so have a special use in the delivery of certain pharmaceutical and biologic products, specifically those that lose potency, are subject to degradation when exposed to moisture, or otherwise alter state rapidly after mixing or reconstitution. Certain drugs must be freeze dried (lyophilized) to be held in a stable state for sufficient time to make them commercially viable and housed in a container that will allow the reconstitution with a solvent just prior to use. Following reconstitution the resulting liquid must be injected within a specified period of time, after which its physical properties alter or the chemical composition no longer produces the desired effect. A variety of currently marketed drugs require, or the manufacturers chose to use this lyophilization and reconstitution process to maintain sterility, stability and allow for easier handling. Examples of products that have used and/or are using a lyophilization system include: Genotropin (human growth hormone); Humatrope (human growth hormone); Betaseron (interferon); GlucaGen (human recombinant glucagon), Glucagon Emergency System (Eli Lilly) and Activase (alteplase). In the book titled Lyophilization of Biopharmaceuticals by Henry R. Costantino, published in 2004, the author explains, starting at page 444, the rationale for lyophilized drugs and the reasons why using a lyophilization and reconstitution process may be necessary.

Glucagon is a product that generally requires lyophilization to create a stable powder form of the drug that is reconstituted just prior to use. Indeed, the reconstituted product generally must be injected within hours or days, or it changes physical state and potency, rendering it unusable. However, even though lyophilized glucagon is readily available, and dual chamber cartridges containing glucagon have been proposed, see e.g. PCT Publication No. WO 07/075,839, published 5 Jul. 2007, no glucagon-containing, dual cartridge system has been successfully developed. One potential limitation on the use of dual chamber cartridge technology for the administration of glucagon together with an auto-injector, is that this technology requires a formulation of the drug that reconstitutes rapidly, with 10 seconds or less being ideal, once the cartridge is activated without the need for agitation. This issue is critical not only because dissolution and reconstitution time are critical in an emergency situation but also because administration of incompletely dissolved, reconstituted glucagon may fail to have the therapeutic effect needed.

The present invention solves this problem by providing lyophilized formulations of glucagon that reconstitute rapidly in dual chamber cartridges and so enables new medical devices to meet this important, unmet need for simpler, more reliable devices to administer glucagon to diabetic patients. This same process could be used with a subset of other drugs requiring lyophilization and rapid reconstitution.

### SUMMARY OF THE INVENTION

In some implementations of the present invention, a pharmaceutical formulation is provided comprising glucagon. Some implementations of the present invention further comprise a bulking agent or matrix builder which provides the structure of the lyophilized product. Bulking agents are of particular importance when an active ingredient of a pharmaceutical formulation (i.e. glucagon) is in a low concentration (<1%). In some instances, a bulking agent of the present invention forms a matrix which provides the mass to hold the glucagon and form the suitable cake appearance. Some formulations of the present invention may also provide one or more bulking agents that protect the glucagon or other active pharmaceutical ingredient (API) from degradation and/or potency loss due to the lyophilization and reconstituting conditions. Bulking agents of the present invention may include one or more carbohydrates, amino acids, salts, mannitol, lactose, and sodium chloride. Some pharmaceutical formulations of the present invention further include an acidifying agent selected from either phosphoric acid or hydrochloric acid in a pharmaceutically acceptable diluent, such as sterile water. In other implementations, a pharmaceutical formulation is provided comprising glucagon, an acidifying agent in a pharmaceutically acceptable diluent, such as sterile water, and a bulking agent including at least one of mannitol, lactose, or sodium chloride.

In some embodiments, the formulation comprises about 1 mg/ml glucagon (or approximately 91% (w/v)), hydrochloric acid or phosphoric acid to achieve a pH between 2.5 and 3.0, and 0.5-1.0% mannitol. In other embodiments, the formulation comprises about 1 mg/ml glucagon, an acidifying agent to achieve a pH between 2.5 and 3.0, and 0.5-1.0% lactose or other bulking agent. Generally, formulations of the present invention are suitable for lyophilization (freeze drying) as described herein. In some embodiments, a glycerin-free, glucagon formulation is provided.

In other implementations, a glucagon formulation is provided in a lyophilized form, which is termed a “cake” or “powder” and is physically stable and compatible with a pharmaceutically acceptable solvent mixture. Methods for forming this cake are also provided by the invention.

Further, in some instances the present invention provides a dual chamber cartridge containing the lyophilized cake. In some embodiments, the dual chamber cartridge contains the cake in one chamber and a solvent in the other. In some instances, the solvent can be introduced into the chamber containing the lyophilized glucagon cake such that the reconstitution of the glucagon cake is accomplished in less than 10 seconds. In some instances, reconstitution of the glucagon cake is achieved in approximately 5-7 seconds. This speed and the complete dissolution and reconstitution provided by various embodiments of the present invention are optimal for the delivery of the glucagon liquid using an auto-injector matched to the cartridge in the treatment of severe (emergency) hypoglycemia.

Further still, in some implementations of the present invention a solvent for reconstitution of the glucagon cake is provided. In some instances, the solvent includes a diluent comprising sterile water, at least one of hydrochloric acid or phosphoric acid to achieve and maintain a pH of between approximately 2.5 and 3.0, and mannitol at a concentration of approximately 4.5% (w/v). In some instances, the present invention provides a formulation of glucagon prepared by reconstituting the lyophilized glucagon cake of the invention in a solvent of the invention. In some implementations, neither the solvent nor the reconstituted glucagon formulation contains any glycerin (i.e. glycerin-free).

In some instances, the present invention provides a cartridge having a first and second chamber, wherein one chamber contains the glucagon cake, and the other chamber contains the solvent. In some instances, the cartridge comprises an auto-injector device that can be used to initiate the mixing process and activate the needle and cartridge. For these embodiments, the cartridge provides ease of injection of the completed mixture. In other embodiments, the invention provides a cartridge resulting from mixing the contents of the two chambers of a dual chamber cartridge of the invention, e.g., a cartridge comprising the reconstituted glucagon formulation of the invention.

The present invention greatly simplifies the glucagon preparation and injection process. Some embodiments of the present invention further improve the safety of quickly administering the reconstituted glucagon formulation. For example, some embodiments of the present invention provide a lyophilized glucagon formulation and a solvent each housed within their own distinct chamber within a dual chamber cartridge. The dual chamber cartridge facilitates rapid initiation of the mixing process. In some instances, the mechanical activity or physical movement of the cartridge or pen unit is sufficient to reconstitute the glucagon formulation. Further, some cartridges comprise a pen design whereby the needle is shield prior to, and after injection of the reconstituted glucagon formulation. Thus, some embodiments of the present invention provide a medical device which eliminates user error and minimizes potential for both primary and secondary needle sticks. Accordingly, the present invention provides a medical device that is suited to the mixing and administration time constraints common to emergency situations such as severe hypoglycemia.

These and other aspects and embodiments of the invention are described in more detail below.

### DETAILED DESCRIPTION OF THE INVENTION

The pharmaceutical formulations of the present invention have been developed in response to the present state of the art, and in particular, in response to the problems and needs in the art that have not been fully solved by currently available pharmaceutical formulations.

As used herein, the term “acidifying agent” is understood to include any pharmaceutically acceptable acid or combination of acids that are added to a pharmaceutical formulation to achieve a desired pH. Non-limiting examples of acidifying agents include hydrochloric acid, and phosphoric acid.

As used herein, the term “bulking agent” is understood to include any pharmaceutically acceptable additive or matrix builder that may be added to a pharmaceutical formulation as a vehicle for lyophilized preparations. Bulking agents may further include additives which are provided as stabilizers and/or tonicifiers for the pharmaceutical formulation. Non-limiting examples of bulking agents include carbohydrates, amino acids, salts, mannitol, lactose, sucrose, dextran, sodium chloride, and combinations thereof.

In accordance with the invention as embodied and broadly described herein in the preferred embodiment, a pharmaceutical formulation containing glucagon is provided. In some embodiments, the present invention provides a formulation comprising glucagon, mannitol, and an acidifying agent, such as hydrochloric acid or phosphoric acid in a pharmaceutically acceptable diluent, such as sterile water, wherein the formulation is suitable for lyophilization in a dual chamber cartridge such as the Vetter VL-K system. Other embodiments of the present invention include a pharmaceutical formulation comprising glucagon, a bulking agent selected from mannitol, lactose, sucrose, dextran, sodium chloride, or combinations thereof, and an acidifying agent. In some embodiments, the formulation comprises 2 mg/ml glucagon, hydrochloric acid or phosphoric acid in a concentration to achieve a pH between 2.5 and 3.0 (i.e., pH 2.5), and 0.5-2% mannitol. For example, some formulations of the present invention are prepared in a first (lyophilization) chamber of a dual chamber cartridge by adding 0.55 ml of 2 mg/ml glucagon (pH adjusted to between 2.5 and 3.0 with either hydrochloric acid or phosphoric acid) in 1% mannitol (5.5 mg in 0.55 ml). In some embodiments, the pharmaceutical formulation comprises approximately 1 mg of glucagon. In other embodiments, the pharmaceutical formulation comprises glucagon in a final concentration of approximately 91.0% (w/v) (i.e. 1 mg of glucagon into 1.1 ml of solvent).

Glucagon is a 29 amino acid peptide. Besides being naturally produced by the human body, it can also be prepared using recombinant technology or by chemical synthesis. Synthetic glucagon is currently manufactured by three principal suppliers (Bachem AG of Bubendorf, Switzerland, catalog number H-6790.0001, American Peptide, catalog number 1946-1-26A, and AmbioPharm, catalog number [reference]). All three synthetic glucagons are cGMP compliant.

Both recombinant glucagons are marketed in the United States and in other countries. One of these is produced by growing a modified Saccharomyces cerevisiae strain and purifying the resulting compound. This human recombinant glucagon is marketed; the marketed product includes two vials: one in which 1 mg of glucagon, modified by the addition of lactose, hydrochloric acid, and sodium hydroxide, has been lyophilized, and a second vial that contains sterile water for reconstitution of the glucagon. The other recombinant glucagon is grown using a modified Escherichia coli strain. The marketed version of this human recombinant glucagon is similar to the other in that it has lactose and hydrochloric acid added prior to lyophilization, which occurs within a first vial, and the solvent (in a second vial) is a combination of sterile water, glycerin and hydrochloric acid.

To prepare some embodiments of a glucagon formulation suitable for lyophilization in accordance with the invention, glucagon, such as the Bachem synthetic glucagon, at a concentration of 2 mg/ml in 1% mannitol (in sterile water), is adjusted to pH 2.5 to 3.0 (typically pH 2.5) with either hydrochloric or phosphoric acid, thereby providing a solution or glucagon formulation that is suitable for lyophilization. The resulting formulation may then be lyophilized (freeze dried), for example by placing an aliquot of the glucagon formulation in a first chamber of a dual chamber device. In some instances, 0.55 ml of this solution or formulation is placed into a first chamber of a dual chamber device. Thus, some embodiments of the present invention provide a lyophilized form of the glucagon formulation, which lyophilized form is termed a “cake” or “powder” and is physically stable and compatible with a pharmaceutically acceptable solvent mixture.

Some embodiments of the present invention further provide a solvent for reconstitution or dissolving of the lyophilized glucagon cake. In some instances, a suitable solvent comprises a pharmaceutically acceptable diluent (such as sterile water), and mannitol at a concentration of approximately 4.5% (w/v). The pH of the solvent is further adjusted to between approximately 2.5 and 3.0 (i.e., pH 3.0) with at least one of phosphoric or hydrochloric acid. In some embodiments, approximately 1.1 ml of this formulation is added to the second chamber of a dual chamber cartridge.

In some embodiments, the present invention provides a dual chamber cartridge containing the lyophilized glucagon cake in a first chamber, and the pharmaceutical solvent in a second chamber. In some embodiments, the solvent is introduced into the chamber containing the glucagon cake to dissolve or reconstituted the glucagon cake in less than 10 seconds. In other embodiments, the cake is dissolved in less than 10 seconds with an average of 5 to 7 seconds. This speed and the complete dissolution and reconstitution provided by this embodiment are optimal for the rapid delivery of the glucagon liquid in the treatment of severe (emergency) hypoglycemia.

In some instances, a ratio of mannitol (in cake vs. solvent) is important. Excess mannitol in the lyophilized glucagon cake will increase the reconstitution time. Additionally, the breakloose and gliding force of the dual chamber cartridge is increased to a point where the pen is not useable due to insufficient force by the cartridge's spring. The concentration of mannitol in the second chamber ensures that the final formulation injected is isotonic, thereby minimizing the potential for causing pain and tissue damage to the patient. In some instances, the second chamber contains approximately 1.1 ml of solvent thereby ensuring that at least 1 ml of final formulation is administered to the patient.

In some embodiments, a cartridge is provided containing the glucagon cake and solvent housed in an auto-injector device that can be used to initiate the mixing process and activate the needle and cartridge, thereby providing ease of injection of the final formulation. If a dual chamber cartridge is employed, then it may be assembled into an auto-injector pen device in which it can be mixed immediately prior to use and injection. The glucagon formulation is lyophilized in one half of the dual chamber cartridge using a formulation of the invention that allows for a proper cake formation, stability, and rapid reconstitution time, as provided herein.

Lyophilization is a well known process used to produce injectable products that are not stable in solution. The process is performed in equipment called a lyophilizer (essentially a container that can be frozen and put under vacuum) and consists of four steps: (1) freezing the solution slowly by lowering the temperature; (2) slowly raising the temperature to about 0 degrees C. while the frozen solution is under vacuum (primary drying); (3) raising the temperature to about room temperature while the material is under vacuum (secondary drying); and (4) removing the vacuum with filtered nitrogen. For products in dual chamber cartridges, the freezing time is about 5-20 hours, and the lowest freezing temperature is about minus 55 degrees C. The final lyophilized cake will have a moisture content of under 5%, and typically between 0.5-3%.

After the lyophilization process, the chamber holding the dried or lyophilized drug is then sealed with an elastomeric closure. Then the specially formulated solvent of the invention is added to the second chamber (to work in conjunction with the lyophilized powder) and an elastomeric stopper added to seal the end holding the liquid. The resulting cartridges can then be shipped, for example to a location for assembly into the auto-injector device. The cartridge is placed inside the auto-injector followed by attaching the needle assembly to complete the device. The completed device is then placed in a container to hold and protect the device as well as the necessary literature and instructions.

An auto-injector is a medical device designed to deliver a single dose of a particular drug. Some auto-injectors contain spring-loaded syringes. By design, auto-injectors are intended for ease of use and are compatible for self-administration by patients, or administration by caregivers in circumstances when the patient is unable to self-administer, such as severe hypoglycemia. The site of injection depends on the drug loaded. The injectors were initially designed to overcome the hesitation associated with self-administration of the needle-based drug delivery device. Examples of auto-injector devices include: EpiPen; Rebiject; Aranesp; and Enbrel. See EP Patent Pub. No. 1 743 666 (Scandinavian Health Limited), incorporated herein by reference.

A suitable auto-injector device for purposes of the present invention will generally house a dual chamber cartridge containing lyophilized glucagon in one chamber and a solvent in the second chamber. Both the lyophilized drug and the solvent use the formulations provided herein to facilitate rapid reconstitution and administration. In addition, the auto-injector either includes or is compatible with a needle assembly.

Existing auto-injectors, whether using a lyophilized dual chamber cartridge or an existing liquid, require the attachment and activation of a needle prior to each use. Under current applications, a sterile needle, housed in a container covering the needle and attachment end, must be held in one hand while the protective cover is removed. The user then presses the threaded (typically female) end of the needle assembly against the opposite (typically male) end of the device followed by a twisting motion such that the female end is threaded onto the make end using the necessary number of turns. Other needle products accomplish the same result with snap on, rather than threaded fastening systems. This allows the base end of the needle to puncture or enter the unit holding the liquid or reconstituted drug allowing for an injection to occur. The protective cover over the needle is removed and the injection is given. This presents a needle that is exposed and may accidentally pierce the skin of the person receiving the injection as well as the person delivering (or any other party in proximity of) the injection.

Exposed needles, used needles, and contaminated needles, can transmit various diseases including but not limited to hepatitis, HIV, infections and others. Secondary needle stick (piercing of a second person's skin after administering an injection to the primary party) is a serious health risk. In certain embodiments of the invention, the needle assembly is attached to the auto-injector and is pre-threaded, or has a snap-on mechanism for ease of attachment. The needle is further protected by a shield. The person preparing the injection activates the auto-injector (“pen”) by turning the distal (non-needle end) which allows an internal spring to press a rod into the solvent chamber end of the cartridge which subsequently forces the solvent toward the chamber holding the lyophilized glucagon where reconstitution occurs. The rotation (turning) of the needle cover allows the base of the needle assembly to puncture the end of the cartridge containing the reconstituted glucagon while concurrently allowing the device barrel to move slightly forward to hide the needle as the needle cover is removed. This allows for the needle to be hidden and shielded prior to the injection.

In some embodiments, it is important that the needle not be allowed to puncture the cartridge prior to the reconstitution of the glucagon; otherwise, the mixing within the cartridge could be compromised as a result of the liquid passing into the needle prior to mixing. If the needle punctures the cartridge prematurely, moisture may be allowed to enter into the first chamber, or the chamber containing the lyophilized glucagon formulation, which could compromise the integrity of the drug. Further, premature wetting to the glucagon formulation may result in bacterial contamination and/or allow the drug to degrade leading to insufficient effect for the condition.

A pre-attached needle assembly is valuable, because of the nature of the emergency situation (severe hypoglycemia) that can be treated with the formulations of the invention. The patient often cannot prepare and administer the injection without assistance. The patient must often rely on a second person to locate the product, and prepare and administer the injection. The requirement to find a needle to attach, and the potential for secondary needle stick (and contamination or injury) due to an exposed needle, are avoided with a system in which the needle is attached to the auto-injector but hidden and shielded prior to and after the injection.

Thus, in one embodiment, the present invention provides a “pen” style device. Specifically, the “pen” device is custom manufactured to hold the dual chamber cartridge, to mix the lyophilized glucagon and solvent properly, and to facilitate injection of the reconstituted glucagon. In one embodiment, the “pen” device has a pre-attached needle unit that is “activated” immediately subsequent to mixing and prior to injection. The base end of this special needle unit punctures the proximal (upper) end of the cartridge that houses the reconstituted glucagon. Pressing the needle shield of the “pen” against the skin triggers the spring-loaded injection to occur in that the reconstituted glucagon liquid is forced through the needle and into the intramuscular area where the glucagon is absorbed.

Various embodiments of the glucagon formulations, dual chamber cartridges, and pen delivery system of the present invention meet the following criteria. The pen delivery system is configured to achieve the same result as the current emergency glucagon products on the market (e.g. Novo and Lilly), except that the glucagon lyophilized cake and solvents are in the dual chambers of a cartridge instead of in vials, and are mixed automatically by the auto-injector rather than manually by hand. Thus, in some embodiments a first chamber having a fill volume of 1.1 ml contains the lyophilized glucagon cake, and a second chamber having a fill volume of 1.1 ml contains the solvent. In the pen delivery system of the invention, the dissolution of the lyophilized glucagon powder in the first chamber after reconstitution with solvent from the second chamber is simple, rapid, and takes less than 15 seconds without requiring any major agitation or shaking. In some instances, the dissolution of the lyophilized glucagon powder is achieved in less than 10 seconds. In some embodiments, the dissolution of the lyophilized glucagon powder is achieved in approximately 5-7 seconds.

Some embodiments of the present invention further provide a dual chamber containing the formulations disclosed herein, wherein the dual chamber breakloose and gliding force of the individual chamber plungers are similar to that of an empty cartridge. In some instances, the breakloose and gliding forces are lower than that of an empty cartridge. Thus, various embodiments of the present invention may be used with a pen delivery or auto-injector device that is currently on the market, or which may easily obtain 510K approval. These and other aspects and embodiments of the invention are described in the following Example.

### EXAMPLES

This example describes several sets of experiments demonstrating the advantages and properties of the glucagon formulations of the invention.

In the first set of experiments, formulations (containing 5% mannitol) similar to the marketed Novo emergency glucagon products were studied. Two different fill volumes (0.55 ml and 1.1 ml) in a first chamber (1) of a dual chamber cartridge were studied. The experiments were designed to evaluate the cake appearance and reconstitution time of lyophilized cakes of different sizes. In addition, two different acids, phosphoric acid (H3PO4, non-volatile) and hydrochloric acid (HCl, volatile), were used to adjust the solution to pH 2.5 to aid the dissolution of the glucagon. The pH changes after reconstitution were measured.

A typical lyophilization cycle for 5% mannitol was used (about 20 hours of primary drying and 10 hours of secondary drying). Thermocouples were placed in different areas in the same rack (corner and middle) and in different racks (top, middle and bottom) to measure the temperature and monitor lyophilization progress. After lyophilization of chamber 1, and filling a second chamber (2) with water for injection (WFI), cartridge samples from different areas in the same rack (corner, middle) and from different racks (top, middle and bottom) were analyzed for appearance, moisture level of the lyophilized cake, reconstitution time, pH, and osmolarity after reconstitution. Breakloose and gliding forces were also measured. Placebo cartridges were also used to assess the lyophilization cycle and serve as a control. Typical test results for the active and placebo cartridges placed at the corner of the racks are shown in Table 1 and Table 2, respectively. The breakloose and gliding forces for empty cartridges were also measured as a control (data not shown).

TABLE 1
Effects of different fill volumes in Chamber 1 and pH adjusting
acids on the properties of the Emergency Glucagon Drug Products

| | A1 | A2 | B1 | B2 |
|---|---|---|---|---|
| Chamber 1 | 1.1 ml of 1 | 0.55 mL of 2 | 1.1 ml of 1 | 0.55 mL of 2 |
| | mg/ml glucagon | mg/ml glucagon | mg/ml glucagon | mg/ml glucagon |
| | in 5% mannitol, | in 10% mannitol, | in 5% mannitol, | in 10% mannitol, |
| | pH 2.5 adjusted | pH 2.5 adjusted | pH 2.5 adjusted | pH 2.5 adjusted |
| | with HCl | with HCl | with phosphoric | with phosphoric |
| | | | acid | acid |
| Chamber 2 | 1.1 ml water for | 1.1 ml WFI | 1.1 ml WFFI | 1.1 ml WFI |
| | Injection (WFI) | | | |
| Test results | | | | |
| Appearance | Intact cake at | Intact cake at | Intact cake at | Intact cake at |
| | day 0 and some | day 0 and some | day 0 and some | day 0 and some |
| | shrinkage in 7- | shrinkage in 7- | shrinkage in 7- | shrinkage in 7- |
| | 11 days at 2-8 | 11 days at 2-8 | 11 days at 2-8 | 11 days at 2-8 |
| | degrees C. | degrees C. | degrees C. | degrees C. |
| pH increases after | 0.5 | 0.7 | <0.1 | 0.3 |
| reconstitution | | | | |
| Moisture (%) at the | 2.13 ± 1.1 | 2.12 ± 0.01 | 3.55 ± 0.54 | 2.92 ± 0.10 |
| corner of the racks | | | | |
| Reconstitution time | 40 ± 15 | 24 ± 3 | 37 ± 7 | 24 ± 3 |
| (seconds) at the | | | | |
| corner of the racks | | | | |
| Osmolarity (mOsmol/kg, | 282 | 281 | 292 | 289 |
| average of 3) | | | | |
| Maximum Break loose | 158 | 58 | 30 | 15 |
| force (N) | | | | |
| Mean Break loose | 95 | 48 | 23 | 14 |
| force (N) | | | | |
| Maximum of maximum | 150 | 58 | 27 | 18 |
| gliding force (N) | | | | |
| Mean of maximum | 77 | 53 | 23 | 14 |
| gliding force (N) | | | | |
| Mean of average | 31 | 27 | 14 | 9 |
| gliding force (N) | | | | |

TABLE 2
Effects of different fill volumes in Chamber 1 and pH adjusting acids
on the properties of the Emergency Glucagon Placebo Products

| | A1 | A2 | B1 | B2 |
|---|---|---|---|---|
| Chamber 1 | 1.1 ml of 5% | 0.55 mL of 10% | 1.1 ml of 5% | 0.55 mL of 10% |
| | mannitol, pH | mannitol, pH | mannitol, pH | mannitol, pH |
| | 2.5 adjusted | 2.5 adjusted | 2.5 adjusted | 2.5 adjusted |
| | with HCl | with HCl | with phosphoric | with phosphoric |
| | | | acid | acid |
| Chamber 2 | 1.1 ml water for | 1.1 ml WFI | 1.1 ml WFFI | 1.1 ml WFI |
| | Injection (WFI) | | | |
| Test results | | | | |
| Appearance | Intact cake at | Intact cake at | Intact cake at | Intact cake at |
| | day 0 and some | day 0 and some | day 0 and some | day 0 and some |
| | shrinkage in 7- | shrinkage in 7- | shrinkage in 7- | shrinkage in 7- |
| | 11 days at 2-8 | 11 days at 2-8 | 11 days at 2-8 | 11 days at 2-8 |
| | degrees C. | degrees C. | degrees C. | degrees C. |
| pH increases after | 0.8 | 0.9 | <0.1 | 0.3 |
| reconstitution | | | | |
| Moisture (%) at the | 1.86 ± 0.05 | 1.71 ± 0.73 | 2.10 ± 0.33 | 2.78 ± 0.81 |
| corner of the racks | | | | |
| Reconstitution time | 92 ± 41 | 56 ± 8 | 58 ± 8 | 97 ± 43 |
| (seconds) at the | | | | |
| corner of the racks | | | | |
| Osmolarity (mOsmol/kg, | 282 | 289 | 290 | 286 |
| average of 2) | | | | |
| Maximum Break loose | 160 | 147 | 34 | 61 |
| force (N) | | | | |
| Mean Break loose | 150 | 137 | 32 | 43 |
| force (N) | | | | |
| Maximum of maximum | 104 | 149 | 43 | 59 |
| gliding force (N) | | | | |
| Mean of maximum | 79 | 141 | 27 | 30 |
| gliding force (N) | | | | |
| Mean of average | 35 | 30 | 16 | 12 |
| gliding force (N) | | | | |

From the first set of experiments (results summarized above in Tables 1 and 2), several conclusions were drawn. The lyophilization cycle for the different 5% mannitol formulations (at two different fill volumes of 1.1 ml and 0.55 ml and with two different acids, hydrochloric and phosphoric acids) produced acceptable cake, with the right moisture level (<5%) and osmolarity (260-320 mOsmol/kg). The reconstitution time for the active cartridges is less than that of the placebo cartridges (less than 1 minute versus over 1 minute). The active cartridges with the smaller cake size (0.55 ml) had the fastest reconstitution time (about 24 seconds). In addition, pH adjustment with either hydrochloric acid or phosphoric acid was acceptable, except there was some small loss of hydrochloric acid during lyophilization. The breakloose and gliding forces for all the cartridges and formulations were too high, except for the cartridges containing formulation B2 (0.55 ml cake size and adjusted with phosphoric acid). The B2 formulation has similar breakloose and gliding forces to the empty cartridges.

Based on the results of the first set of experiments, a second set of experiments to evaluate only one fill volume for chamber 1 (0.55 ml), different mannitol concentrations in chamber 1, different mannitol concentrations in chamber 2 and the two acids, hydrochloric and phosphoric using a similar lyophilization cycle. Slightly different primary and second drying cycle times were used. The cartridges were tested as described above for the results summarized in Table 1. The results are shown in Table 3.

TABLE 3
Effects of different mannitol concentrations in a first chamber (1) and
pH adjusting acids on the properties of the Emergency Glucagon Products

| | A2 | B2 | A3 | B3 | A4 | A5 | B5 |
|---|---|---|---|---|---|---|---|
| Chamber 1 | 10% | 10% | 4% | 4% | 3% | 2% | 2% |
| (0.55 ml of 2 | mannitol, | mannitol, | mannitol, | mannitol, | mannitol, | mannitol, | mannitol, |
| mg/ml | pH 2.5 | pH 2.5 | pH 2.5 | pH 2.5 | pH 2.5 | pH 2.5 | pH 2.5 |
| glucagon with | adjusted | adjusted | adjusted | adjusted | adjusted | adjusted | adjusted |
| various mannitol) | with HCl | with H3PO4 | with HCl | with H3PO4 | with HCl | with HCl | with H3PO4 |
| Chamber 2 | WFI | WFI | 3% | 3% | 3.5% | 4% | 4% |
| (1.1 ml) | | | mannitol | mannitol | mannitol | mannitol | mannitol |
| Test results | | | | | | | |
| Appearance | Intact cake | Intact cake | Intact cake | Intact cake | Intact cake | Intact cake | Intact cake |
| | at day 0 and | at day 0 and | at day 0 and | at day 0 and | at day 0 and | at day 0 and | at day 0 and |
| | some shrinkage | some shrinkage | no shrinkage | no shrinkage | no shrinkage | no shrinkage | no shrinkage |
| | in 4 days at | in 4 days at | overtime at | overtime at | overtime at | overtime at | overtime at |
| | 2-8 degrees C. | 2-8 degrees C. | 2-8 degrees C. | 2-8 degrees C. | 2-8 degrees C. | 2-8 degrees C. | 2-8 degrees C. |
| pH increases | 0.6 | 0.5 | 0.8 | 0.5 | 0.9 | 0.9 | 0.4 |
| after | | | | | | | |
| reconstitution | | | | | | | |
| Moisture (%) | 2.66 ± 0.99 | 2.76 ± 1.38 | 1.17 ± 0.38 | 2.21 ± 0.01 | 1.02 ± 0.43 | 0.54 ± 0.05 | 1.01 ± 0.25 |
| at corner of | | | | | | | |
| racks | | | | | | | |
| Reconstitution | 45 ± 10 | 49 ± 9 | 14 ± 4 | 12 ± 2 | 15 ± 9 | 15 ± 4 | 12 ± 2 |
| time | | | | | | | |
| (seconds) at | | | | | | | |
| corner of | | | | | | | |
| racks | | | | | | | |
| Osmolarity | 282 | 290 | 281 | 283 | 284 | 287 | 288 |
| (mOsmol/kg, | | | | | | | |
| average of 3) | | | | | | | |
| Maximum | 28.7 | 21 | 69.7 | 33.6 | 26.4 | 18.7 | 16.6 |
| Break loose | | | | | | | |
| force (N) | | | | | | | |
| Mean Break | 20.8 | 17.4 | 52.9 | 29.7 | 20.3 | 15.8 | 13.6 |
| loose force (N) | | | | | | | |
| Maximum of | 42.6 | 32.4 | 37.4 | 26 | 21.6 | 16 | 15 |
| maximum | | | | | | | |
| gliding force (N) | | | | | | | |
| Mean of | 31.2 | 23.0 | 30.5 | 22.9 | 19.3 | 14.3 | 12.9 |
| maximum | | | | | | | |
| gliding force (N) | | | | | | | |
| Mean of | 18.6 | 14.4 | 21.0 | 17.6 | 12.4 | 9.4 | 8.7 |
| average | | | | | | | |
| gliding force (N) | | | | | | | |

The results for the second set of experiments (summarized in Table 3, above), clearly demonstrated that using 0.55 ml of 2 mg/ml glucagon in 2% mannitol in a first chamber produced product with good cake appearance and with both osmolarity and moisture meeting specifications of 260-320 mOsmol/kg and not more than 5%, respectively. The breakloose and gliding forces were similar to that of the empty cartridges. However, after lyophilization, there was a higher loss of hydrochloric acid than phosphoric acid. This loss of hydrochloric acid was not significant, and because hydrochloric acid has been demonstrated to be safe for human administration in glucagon formulations, hydrochloric acid is preferred for use in pH adjustment and to aid in the dissolution of glucagon. However, using phosphoric acid for pH adjustment, the products appear to have slightly faster dissolution, lower breakloose and gliding force as well as lower loss of the acid after lyophilization.

In the next set of experiments, the effects of 1% and 2% mannitol (0.55 ml) and placebo in chamber 1 on the properties of glucagon were studied. In addition, a fill volume of 1.1 ml of 1% mannitol in chamber 1 was evaluated. The lyophilization cycle was similar. The test results are shown in Table 4.

TABLE 4
Effects of 1% and 2% mannitol in Chamber 1 on the properties of the Emergency glucagon active and placebo Products

| | A5 | A5 | A5 placebo | A6 | A6 | A6 Placebo | A7 |
|---|---|---|---|---|---|---|---|
| Chamber 1 | 0.55 ml of 2 | 0.55 mL of | 0.55 ml of | 0.55 ml of 2 | 0.55 ml of 2 | 0.55 ml of | 1.1 mL of |
| (pH 2.5 | mg/ml | 2 mg/ml | 2% | mg/ml | mg/ml | 1% mannitol | 2 mg/ml |
| adjusted with | glucagon in | glucagon in | mannitol, | glucagon in | glucagon in | | glucagon in |
| HCl) | 2% mannitol | 2% mannitol | | 1% mannitol | 1% mannitol | | 1% mannitol |
| Chamber 2 | 1.1 ml 4% | 1.1 ml 4% | 1.1 ml 4% | 1.1 ml 4.5% | 1.1 ml 4.5% | 1.1 ml 4.5% | 1.1 ml 4% |
| (Adjusted to | mannitol | mannitol | mannitol | mannitol | mannitol | mannitol | mannitol |
| pH 3 with | | | | | | | |
| HCl) | | | | | | | |
| Test results | | | | | | | |
| Appearance | Intact cake | Intact cake | Intact cake | Intact cake | Intact cake | 50% or more | 50% |
| | and no shrinkage | and no shrinkage | and no shrinkage | and no shrinkage | and no shrinkage | cartridges with | cartridges with |
| | at 2-8 degrees C. | at 2-8 degrees C. | at 2-8 degrees C. | at 2-8 degrees C. | at 2-8 degrees C. | collapsed cake | collapsed cake |
| pH increase | 0.32 | 0.26 | 0.31 | 0.39 | 0.23 | 0.52 | 0.37 |
| after | | | | | | | |
| reconstitution | | | | | | | |
| Moisture (%) | 0.85 ± 0.07 | No data | 1.37 ± 0.21 | 0.79 ± 0.05 | 0.79 ± 0.02 | No data | 0.74 ± 0.09 |
| at the corner | | | | | | | |
| of the racks | | | | | | | |
| Reconstitution | 15 ± 3 | 12 ± 4 | 25 ± 6 | 13 ± 10 | 20 ± 8 | 9 ± 2 | 9 ± 3 |
| time | | | | (7 ± 1 from | | | |
| (seconds) at | | | | middle of | | | |
| the corner of | | | | rack) | | | |
| the racks | | | | | | | |
| Osmolarity | 290 | 292 | 290 | 293 | 294 | 290 | 292 |
| (mOsmol/kg, | | | | | | | |
| average of 2) | | | | | | | |
| Maximum | 32.06 | 12.43 | 33.36 | 12.78 | 13.67 | 14.56 | 12.85 |
| Break loose | | | | | | | |
| force (N) | | | | | | | |
| Mean Break | 19.57 | 11.29 | 21.17 | 11.73 | 12.68 | 12.46 | 11.56 |
| loose force (N) | | | | | | | |
| Maximum of | 30.29 | 11.83 | 18.72 | 10.09 | 10.95 | 13.43 | 15.37 |
| maximum | | | | | | | |
| gliding force (N) | | | | | | | |
| Mean of | 18.02 | 10.69 | 15.87 | 9.31 | 9.85 | 11.37 | 12.56 |
| maximum | | | | | | | |
| gliding force (N) | | | | | | | |
| Mean of | 12.12 | 6.9 | 12.06 | 7.13 | 7.63 | 7.9 | 7.68 |
| average | | | | | | | |
| gliding force (N) | | | | | | | |

The results of these experiments (summarized in Table 4, above), clearly showed that the formulation provided by Chamber 1—0.55 ml of 2 mg/ml glucagon in 1% mannitol, pH 2.5 adjusted by hydrochloric acid, and Chamber 2—1.1 ml of 4.5% mannitol, pH 3 adjusted by hydrochloric acid, produced glucagon drug product with: acceptable and intact cake (indicating acceptable and robust lyophilization cycle); low moisture (around 1%); osmolarity of around 290 mOsmol/kg meeting USP specification for isotonic solution (260-320 mOsmol/kg); consistent low reconstitution time (between 10-20 seconds); and low breakloose and gliding forces comparable to empty cartridges.

Because early development runs are performed at a small scale (about 900 cartridges), the results may not be representative of cartridges manufactured at a larger scale. Therefore, several large scale runs (at commercial scale of 61000 cartridges) were performed using the formulation above. Because the breakloose and gliding forces for the glucagon product may increase during storage, the siliconization level of the cartridges for these runs was increased from 0.3 mg/cartridge to 0.6 mg/cartridge. Thus, in one embodiment, the dual chamber cartridge of the invention has a siliconization level higher than 0.3 mg/cartridge, including but not limited to 0.6 mg/cartridge. Only about 4000 active cartridges were filled; about 57000 placebo cartridges were included to simulate the commercial batch size process. Test results are shown in Table 5 for two simulated full scale runs. The test results showed consistency with the earlier, smaller-scale test data: intact and homogeneous cake; pH after reconstitution at around 2.9; moisture of lyophilized cake at around 1% or less; osmolarity around 300 mOsmol/kg; reconstitution time of around 5-7 seconds; and breakloose and gliding forces are low and similar to the empty cartridges.

The data of Table 5 supports a conclusion that low moisture level may be related to the short reconstitution time; thus, some embodiments of the present invention provide lyophilized glucagon in which the moisture content is 0.1 to 1%.

TABLE 5
Full scale run effect on the properties of the Emergency glucagon Product

| | Full scale Run 1 | | Full Scale run 2 | |
|---|---|---|---|---|
| Chamber 1 | 0.55 ml of 2 mg/ml | | 0.55 ml of 2 mg/ml | |
| | glucagon in 1% | | glucagon in 1% | |
| | mannitol, pH 2.5 | | mannitol, pH 2.5 | |
| | adjusted with HCl | | adjusted with HCl | |
| Chamber 2 | 1.1 ml 4.5% | | 1.1 ml 4.5% | |
| | mannitol, pH 3.0 | | mannitol, pH 3.0 | |
| Test results | | | | |
| Appearance | Intact and | | Intact and | |
| | homogeneous cake | | homogeneous cake | |
| pH after | 2.92 | | 2.88 | |
| reconstitution | | | | |
| Moisture (%) at the | 0.55 (shelve 1) | 0.54 (shelve 6) | 0.51 (shelve 1) | 0.31 (shelve 6) |
| corner of the racks | 0.35 (shelve 2) | 1.14 (shelve 12) | 0.13 (shelve 2) | 0.96 (shelve 12) |
| (N = 2) | | | | |
| Reconstitution time | 4.4 ± 1.3 | 2.8 ± 0.8 | 5 ± 0 | 5 ± 1.4 |
| (seconds) at the | (shelve 1) | (shelve 6) | (shelve 1) | (shelve 6) |
| corner of the racks | 4 ± 1.4 | 3.2 ± 0.4 | 6.5 ± 2.1 | 4 ± 0 |
| | (Shelve 2) | (Shelve 12) | (Shelve 2) | (Shelve 12) |
| Osmolarity (mOsmol/kg, | 300 | | 297 | |
| average of 3) | | | | |
| Maximum Break loose | 10.62 | | 10.5 | |
| force (N) | | | | |
| Mean Break loose | — | | — | |
| force (N) | | | | |
| Maximum of maximum | 9.7 | | 10.2 | |
| gliding force (N) | | | | |
| Mean of maximum | — | | — | |
| gliding force (N) | | | | |
| Mean of average | 5.81 | | 6 | |
| gliding force (N) | | | | |

It is underscored that the present invention may be embodied in other specific forms without departing from its spirit or essential characteristics. Therefore, the described embodiments herein should be deemed only as illustrative. These and other aspects and embodiments of the invention are described in the following claims.

1. A pharmaceutical formulation comprising glucagon, a bulking agent, and an acidifying agent in a pharmaceutically acceptable diluent.

2. The pharmaceutical formulation of claim 1, further comprising a glucagon formulation suitable for lyophilization, and a solvent for dissolving the lyophilized glucagon formulation.

3. The pharmaceutical formulation of claim 2, wherein the glucagon formulation comprises approximately 1 mg of glucagon, and wherein the bulking agent is mannitol in a concentration from approximately 1.0% to approximately 10.0% (w/v).

4. The pharmaceutical formulation of claim 3, wherein the glucagon formulation further comprises a pH between approximately 2.5 and 3.0.

5. The pharmaceutical formulation of claim 2, wherein the solvent comprises:
sterile water as the pharmaceutically acceptable diluent;
mannitol as the bulking agent in a concentration from approximately 3.0% to approximately 4.5% (w/v); and
the acidifying agent comprises at least one of hydrochloric acid and phosphoric acid at a concentration necessary to achieve a pH for the solvent between approximately 2.5 and 3.0.

6. The pharmaceutical formulation of claim 2, wherein the solvent is configured to dissolve the lyophilized glucagon formulation in less than approximately 10 seconds.

7. The pharmaceutical formulation of claim 1, wherein the pharmaceutically acceptable diluent is sterile water.

8. The pharmaceutical formulation of claim 1, further comprising a glycerin-free pharmaceutical formulation.

9. The pharmaceutical formulation of claim 1, wherein the acidifying agent is at least one of phosphoric acid and hydrochloric acid, and the pharmaceutical formulation further comprises a pH between 2.5 and 3.0.

10. The pharmaceutical formulation of claim 1, further comprising approximately 1 mg of glucagon, and mannitol as the bulking agent in a concentration from approximately 1.0% to approximately 10.0% (w/v).

11. The pharmaceutical formulation of claim 1, further comprising approximately 1 mg of glucagon, and mannitol as the bulking agent in a concentration from approximately 5.0% to approximately 7.0% (w/v).

12. The pharmaceutical formulation of claim 1, further comprising glucagon in a concentration of approximately 91.0% (w/v).

13. A method for rapidly dissolving and administering a glucagon-containing pharmaceutical formulation to a patient, the method comprising:
providing a glucagon formulation suitable for lyophilization, the glucagon formulation comprising approximately 1 mg of glucagon, and a first bulking agent in a concentration from approximately 1.0% to approximately 10.0% (w/v);
lyophilizing the glucagon formulation;
providing a solvent comprising a pharmaceutically acceptable diluent, and a second bulking agent in a concentration of approximately 3.0% to approximately 4.5% (w/v), and a pH between approximately 2.5 and 3.0; and
reconstituting the lyophilized glucagon formulation with the solvent in less than 10 seconds.

14. The method of claim 13, further comprising steps for:
placing the lyophilized glucagon formulation into a first chamber of a dual chamber cartridge; and
placing the solvent in a second chamber of the dual chamber cartridge.

15. The method of claim 14, further comprising a step for adding together the solvent and the lyophilized glucagon formulation to initiate the step of reconstituting the lyophilized glucagon formulation.

16. The method of claim 15, further comprising a step for administering the reconstituted glucagon formulation to the patient.

17. The method of claim 13, wherein the first and second bulking agent is mannitol.

18. A lyophilized pharmaceutical formulation comprising:
approximately 1 mg of glucagon;
a bulking agent in a concentration from approximately 1.0% to approximately 10.0% (w/v); and a pH between approximately 2.5 and 3.0.

19. The formulation of claim 18, further comprising an acidifying agent selected from the group consisting of hydrochloric acid and phosphoric acid in a concentration necessary to achieve the pH.

20. The formulation of claim 18, wherein the bulking agent is mannitol, and the formulation is configured to be completely dissolved by a pharmaceutical solvent in less than approximately 10 seconds.
